(19) United States
(12) Patent Application Publication
(10) Pub. No.: US 2009/0017131 A1
(43) Pub. Date: Jan. 15, 2009

(54) Disinfectant cleaning composition and method

(76) Inventor: Donald S. Peters, Aurora, OH (US)

Correspondence Address: ROETZEL & ANDRESS, 1375 EAST 9TH STREET, CLEVELAND, OH, 44114, US

(21) Appl. No.: 11/827,725

(22) Filed: Jul. 14, 2007

(51) Int. Cl. A61K 33/40; A61P 43/00

(52) U.S. Cl. 424/616

(57) ABSTRACT

Various embodiments of disinfectant cleaning compositions and the method of using them are provided. In one embodiment of the present invention, the disinfectant cleaning composition comprises, by weight, from about 0.5% to about 6% hydrogen peroxide and from about 0.5% to about 20% aloe vera and/or active thereof, and from about 99% to about 74% solvent. In another embodiment, the disinfectant cleaning composition can also include a fragrance.

### FIELD OF THE INVENTION

The present invention relates to the field of surface cleaning compositions for cleaning multiple surfaces, and the method of cleaning surfaces. More specifically, the present invention relates to surface cleaning compositions which include a disinfectant and the method of cleaning surfaces such as countertops.

### BACKGROUND

A variety of surface cleaning formulations are available for household use on hard surfaces such as countertops, tiles, kitchen and bathroom fixtures, for example. Several considerations in formulating such compositions include the cleaning ability to dissolve stains and dirt as well as disinfect the surfaces that they contact.

Despite their convenience, several conventional cleaning formulations suffer from a number of disadvantages. Compositions used on hard surfaces such as countertops, plumbing fixtures in kitchens and bathroom for example and porcelain and plastic plumbing fixtures, for example, are acidic or caustic and can damage the substrates to which they are applied. They can also present safety or health hazards to the user. For example they may irritate the eyes and nose and mucous membranes. Most restaurant kitchens must employ hot water for cleaning surfaces which come into contact with food, however the water may not be hot enough to kill bacteria or prevent its growth or effectively cut grease.

There remains a need for alternative cleaning compositions useful in removing stains and/or odor as well as disinfect surfaces without imparting harmful and toxic exposure to those who use or come into contact with the products.

### SUMMARY

The present invention provides for excellent surface cleaning compositions that can be used on various surfaces for disinfecting and improved cleaning of surfaces while also being safe to use and environmentally benign. The surface cleaning compositions herein are odorless, non-toxic, non-flammable, and bio-degradable.

In one embodiment of the present invention, the disinfectant cleaning composition comprises hydrogen peroxide, aloe vera and/or at least one active ingredient thereof, and solvent. In another embodiment the disinfectant cleaning composition comprises, by weight, from about 0.5% to about 6% hydrogen peroxide and from about 0.5% to about 20% aloe vera and/or active thereof, and from about 99% to about 74% solvent.

In another embodiment, the disinfectant cleaning composition comprises, by weight, from about 0.5% to about 6% to about hydrogen peroxide, from about 0.5% to about 20% aloe vera and/or at least one active ingredient thereof, from about 0.05% to about 5% fragrance, and from about 98.95% to about 69% solvent.

The disinfectant cleaning compositions herein are effective in removing soils and stains including grease and food deposits, while also disinfecting the surface to prevent the growth of bacteria. The compositions are safe to use around children and pets, and are safe for the environment. The above compositions also remain in solution and maintain long shelf life for successful commercial distribution and use.

### DETAILED DESCRIPTION

The disinfectant compositions herein are used to treat a variety of surfaces, for example, hard surfaces typically found in residential or commercial kitchens and bathrooms. Such surfaces include tiles, walls, floors, chrome, glass, smooth vinyl, any plastic, laminated wood, table top, sinks, cook tops, dishes, sanitary fittings used in sinks and showers, shower curtains, wash basins, etc. Hard-surfaces also include household appliances including, but not limited to, refrigerators, freezers, washing machines, automatic dryers, ovens, microwave ovens, dishwashers and so on. Furthermore, the disinfectant compositions are also used for hard-surfaces found in hospitals, restaurants, hotels, public transport vehicles, public pools and showers, and commercial and public laundries for example.

In one embodiment of the present invention, the disinfectant cleaning composition comprises hydrogen peroxide, H₂O₂, aloe vera and/or at least one active ingredient thereof, and at least one solvent. Although not wishing to be bound to any particular active ingredient, it is believed that the active ingredient is at least one or more of the components found in the aloe vera plant. In one embodiment of the present invention, the disinfectant cleaning composition are odorless, non-toxic, non-flammable, and biodegradable.

In another embodiment the disinfectant composition is substantially free of acids, caustics, and bleaches, for example, as well as other substances which may be detrimental to the surface finish or color of the substrate, or detrimental to the health of the user. By the term “substantially free of” it is meant that there is less than about 0.7%, and in another example, less than about 0.3%, and in another examples, less than about 0.05% by weight of a substance in the disinfectant cleaning composition.

Hydrogen peroxide, H₂O₂, can provide certain anti-microbial action against micro-organisms. The hydrogen peroxide is an oxidizer that generates hydroxyl free radicals which attack proteins and nucleic acids within micro-organisms cells. Furthermore, the presence of hydrogen peroxide provides good stain removal capabilites, for example, when applied to a hard surface for cleaning. An advantage of hydrogen peroxide is that it can provide an effervescent action which can irrigate any surface to which it is applied. Hydrogen peroxide can be effective with mechanical flushing or rinsing action once applied to further clean a surface.

Hydrogen peroxide can be provided as a liquid mixture of hydrogen peroxide and water, e.g. as liquid hydrogen peroxide in an aqueous solution. Hydrogen peroxide is commercially available at concentrations of 3%, 6%, 35%, 50%, 70% and 90% in water. For safety, 35% or less hydrogen peroxide solution is commonly used.

Aloe vera which originates from the aloe vera plant, has several constituents. The aloe vera comprises amino acids, anthraquinones, enzymes hormones, lignin, minerals, vitamins, salicylic acid, saponins, sterols and sugars. The anthraquinones include the following: aloe emodin, aloetic acid, aloin, anthracine, antranol, barbaloin, chrysophanic acid, emodin, etheraeal oil, ester of cinnamonic acid, isobarbaloin, resistannol. The anthraquinones are found in the sap. The anthraquinones can provide analgesic, antibacterial, antifungal and antiviral activity, and the primary sap component is aloin/barbalion anthrone derivative. The sugars include monosaccharides (e.g. glucose and fructose) and polysaccarides (e.g. gluco-mannans and polymannose). As stated above, although not wishing to be bound to any particular constituent, it is believed that active ingredient thereof is at least one or more constituent found in aloe vera. In addition, the active ingredient thereof can be natural or synthetic.

Solvent includes, but is not limited to water, alcohol, and mixtures thereof. Solvent can be that which is present in the hydrogen peroxide solution and also that which is added to further dilute a composition containing hydrogen peroxide and aloe vera.

In one embodiment of the invention, the surface cleaning composition comprises, by weight, from about 0.5% to about 6% to about hydrogen peroxide, from about 0.5% to about 20% aloe vera and/or at least one active ingredient thereof, and from about 99% to about 74% solvent. In another embodiment, the surface cleaning composition includes, by weight, from 1% to about 5% hydrogen peroxide, from about 1% to about 5% aloe vera and/or at least one active ingredient thereof, and from about 98% to about 80% by weight solvent. In yet another embodiment, the surface cleaning composition herein can include by weight, from about 2% to about 4% hydrogen peroxide, from about 2% to about 10% aloe vera and/or at least one active ingredient thereof, and from about 96% to about 86% solvent.

In one embodiment of the invention, the surface cleaning composition comprises, by weight, from about 0.5% to about 6% to about hydrogen peroxide, from about 0.5% to about 20% aloe vera and/or at least one active ingredient thereof, and from about 99% to about 74% water. In another embodiment, the surface cleaning composition includes, by weight, from 1% to about 5% hydrogen peroxide, from about 1% to about 5% aloe vera and/or at least one active ingredient thereof, and from about 98% to about 80% by weight water. In yet another embodiment, the surface cleaning composition herein can include by weight, from about 2% to about 4% hydrogen peroxide, from about 2% to about 10% aloe vera and/or at least one active ingredient thereof, and from about 96% to about 86% water.

In another embodiment it may be desirable that the disinfectant cleaning composition further contain a fragrance for use on surfaces which do not come in contact with food. Such surfaces include hard surfaces and soft surfaces such as textiles, fabrics, upholstery, and carpet, for example. Accordingly, in another embodiment of the present invention, the disinfectant surface cleaning composition comprises hydrogen peroxide, H₂O₂, aloe vera and/or at least one active ingredient thereof, at least one fragrance, and at least one solvent. In another embodiment the fragrance is a water-soluble fragrance.

Fragrances selected for use in the disinfectant cleansing compositions of the present invention contains ingredients to provide a fresh impression in the area or on the surface to which the composition is directed. Fragrance material of the disinfectant cleaning composition can include, but are not limited to, aromatic and aliphatic esters having molecular weights from about 130 to about 250; aliphatic and aromatic alcohols having molecular weights from about 90 to about 240; aliphatic ketones having molecular weights from about 150 to about 260; aromatic ketones having molecular weights from about 150 to about 270; aromatic and aliphatic lactones having molecular weights from about 130 to about 290; aliphatic aldehydes having molecular weights from about 140 to about 200; aromatic aldehydes having molecular weights from about 90 to about 230; aliphatic and aromatic ethers having molecular weights from about 150 to about 270; and condensation products of aldehydes and amines having molecular weights from about 180 to about 320, and mixtures thereof. When high initial fragrance impact on fabrics is desired, it is also preferable to select a perfume containing perfume ingredients which are not too hydrophobic. The less hydrophobic perfume ingredients are more soluble in water, and are more available in the freshening composition. Identification of a water-soluble fragrances are well known to those of ordinary skill in the art, and water-soluble fragrances are commercially available from a wide variety of sources.

In one embodiment of the invention, the surface cleaning composition comprises, by weight, from about 0.5% to about 6% to about hydrogen peroxide, from about 0.5% to about 20% aloe vera and/or at least one active ingredient thereof, from about 0.05% to about 5% fragrance, and from about 98.95% to about 69% solvent. In another embodiment, the surface cleaning composition includes, by weight, from 1% to about 5% hydrogen peroxide, from about 1% to about 5% aloe vera and/or at least one active ingredient thereof, from about 0.1% to about 4% fragrance, and from about 97.9% to about 76% by weight solvent. In yet another embodiment, the surface cleaning composition herein can include by weight, from about 2% to about 4% hydrogen peroxide, from about 2% to about 10% aloe vera and/or at least one active ingredient thereof, from about 0.2% to about 2% fragrance and from about 95.8% to about 84% solvent.

In one embodiment of the invention, the surface cleaning composition comprises, by weight, from about 0.5% to about 6% to about hydrogen peroxide, from about 0.5% to about 20% aloe vera and/or at least one active ingredient thereof, from about 0.05% to about 5% water-soluble fragrance, and from about 98.95% to about 69% water. In another embodiment, the surface cleaning composition includes, by weight, from 1% to about 5% hydrogen peroxide, from about 1% to about 5% aloe vera and/or at least one active ingredient thereof, from about 0.1% to about 4% water-soluble fragrance, and from about 97.9% to about 76% by weight water. In yet another embodiment, the surface cleaning composition herein can include by weight, from about 2% to about 4% hydrogen peroxide, from about 2% to about 10% aloe vera and/or at least one active ingredient thereof, from about 0.02% to about 2% water-soluble fragrance and from about 95.8% to about 84% water.

In another embodiment of the present invention the disinfectant cleaning compositions herein may comprise a fragrance which is an essential oil or active thereof. Accordingly, in another embodiment of the present invention, the disinfectant cleaning compositions comprise hydrogen peroxide, aloe vera and/or at least one active ingredient thereof, an essential oil, glycol ether, and solvent.

Essential oils include, but are not limited to, those obtained from thyme, lemongrass, citrus, lemons, oranges, anise, clove, aniseed, geranium, roses, mint, lavender, citronella, eucalyptus, peppermint, camphor, sandalwood, pine, vervain, rosmarin, fleagrass, ratanhiae and cedar and mixtures thereof. Actives of essential oils for use herein include, but are not limited to, thymol (present for example in thyme), eugenol (present for example in clove), menthol (present for example in mint), geraniol (present for example in geranium and rose), verbenone (present for example in vervain), eucalyptol and pinocarvone (present in eucalyptus), cedrol (present for example in cedar), anethol (present for example in anise), carvacrol, hinokitiol, berberine, ferulic acid, methyl salicylic acid, methyl salicylate, terpineol and mixtures thereof.

Glycol ether materials are excellent solvents and coupling agents and are typically miscible with aqueous cleaning compositions of the invention. The boiling points of the materials fall within a range of about 100° C. to about 250° C.

The cleaning composition according to various embodiments of the present invention includes at least one glycol ether (e.g., one or more glycol ethers according to the following formulas R₁—OCH₂CH₂OH, R₁—OCH₂CH₂OCH₂CH₂OH, R₁—OCH₂CH₂O—R₂, where R₁ and R₂ are independently selected from C₁ to C₁₀ linear alkyl groups, C₂ to C₁₀ branched alkyl groups, or even C₁ to C₅ linear alkyl groups, or even C₂ to C₅ branched alkyl groups). Examples of glycol ether include, but are not limited to, propylene glycol ether, dipropylene glycol ether, tripropylene glycol ether, propylene glycol methyl ether, dipropylene glycol methyl ether, dipropylene glycol ethyl ether, tripropylene glycol methyl ether, propylene glycol isobutyl ether, ethylene glycol methyl ether, ethylene glycol ethyl ether, ethylene glycol diethyl ether, ethylene glycol dibutyl ether, diethylene glycol methyl ether, diethylene glycol dimethylether, diethylene glycol ethyl ether, diethylene glycol diethyl ether, diethylene glycol butyl ether, ethylene glycol dimethyl ether and other similar materials, and mixtures thereof.

Therefore, in one embodiment of the invention, the surface cleaning composition comprises, by weight, from about 0.5% to about 6% to about hydrogen peroxide, from about 0.5% to about 20% aloe vera and/or at least one active ingredient thereof, from about 0.05% to about 5% essential oil, from about 0.5% to about 10% glycol ether, and from about 98.45% to about 59% solvent. In another embodiment, the surface cleaning composition includes, by weight, from 1% to about 5% hydrogen peroxide, from about 1% to about 5% aloe vera and/or at least one active ingredient thereof, from about 0.1% to about 4% essential oil, from about 1% to about 5% glycol ether, and from about 96.9% to about 71% solvent. In yet another embodiment, the surface cleaning composition herein can include by weight, from about 2% to about 4% hydrogen peroxide, from about 2% to about 10% aloe vera and/or at least one active ingredient thereof, from about 0.2% to about 1% essential oil, from about 1% to about 3% glycol ether, and from about 94.8% to about 82% solvent. Solvent includes, but is not limited to water, alcohol, and mixtures thereof.

In another embodiment of the present invention, alternate sources for hydrogen peroxide can be used to make various disinfectant cleaning compositions herein. However, depending upon the hydrogen peroxide producing source the disinfectant compositions of the may be designed for use on surfaces other than those which come into contact with food. A hydrogen peroxide producing source can be any compound which produces hydrogen peroxide when the compound is in contact with water. Suitable water-soluble sources of hydrogen peroxide for use herein include, but are not limited to, percarbonates, persilicates, persulphates, such as monopersulfate, perborates and peroxyacids such as diperoxydodecandioic acid (DPDA), magnesium perphthalic acid and mixtures thereof. In another embodiment of the invention, the disinfectant cleaning composition comprises a mixture of hydrogen peroxide and at least one hydrogen peroxide producing source.

In one embodiment of the invention, the surface cleaning composition comprises, by weight, from about 0.5% to about 6% of at least one of hydrogen peroxide and hydrogen peroxide producing source, from about 0.5% to about 20% aloe vera and/or active ingredient thereof, and from about 99% to about 74% water. In another embodiment, the surface cleaning composition includes, by weight, from 1% to about 5% of at least one of hydrogen peroxide and hydrogen peroxide producing source, from about 1% to about 5% aloe vera and/or at least one active ingredient thereof, and from about 98% to about 80% by weight water. In yet another embodiment, the surface cleaning composition herein can include by weight, from about 2% to about 4% of at least one of hydrogen peroxide and hydrogen peroxide producing source, from about 2% to about 10% aloe vera and/or at least one active ingredient thereof, and from about 96% to about 86% water.

In addition, alternative embodiments of the disinfectant cleaning compositions herein can include other classes of peroxides can be used as an alternative to the above hydrogen peroxide and/or producing sources of hydrogen peroxide. Such disinfectant cleaning compositions herein may be used on surfaces, hard or soft surfaces for example, which do not come in contact with food. In another embodiment, these classes of peroxides other than hydrogen peroxide can also be used in combination with hydrogen peroxide and sources of hydrogen peroxide. Suitable classes of peroxides other than hydrogen peroxide can include, but are not limited to, dialkylperoxides, diacylperoxides, preformed percarboxylic acids, organic and inorganic peroxides and/or hydroperoxides. Thus, the disinfectant cleaning composition herein comprise peroxide, aloe vera and/or at least one active ingredient thereof, and solvent. Solvent includes, but is not limited to water, alcohol, and mixtures thereof.

Therefore, in another embodiment of the present invention, the surface cleaning composition comprises, by weight, from about 0.5% to about 6% to about peroxide, from about 0.5% to about 20% aloe vera and/or at least one active ingredient thereof, and from about 99% to about 74% solvent. In another embodiment, the surface cleaning composition includes, by weight, from 1% to about 5% peroxide, from about 1% to about 5% aloe vera and/or at least one active ingredient thereof, and from about 98% to about 80% by weight solvent. In yet another embodiment, the surface cleaning composition herein can include by weight, from about 2% to about 4% peroxide, from about 2% to about 10% aloe vera and/or at least one active ingredient thereof, and from about 96% to about 86% solvent.

The various embodiments of the disinfectant cleaning composition described above can also include one or more optional ingredients. Optional ingredients of the disinfectant cleaning compositions can include all those known by those skilled in the art for the purpose of disinfecting and cleaning, for aesthetics, or otherwise, and depending upon the substrate being cleaned. For substrates which do not come into contact with food, the optional ingredients may include, but are not limited to, disinfectants such as bleaches, for example, peroxygen bleaches and/or chlorine-type bleaches, and for example, aldehydes, glyoxal, parabens, pbiguanide antimicrobial agents, peroxy acids, and mixtures thereof.

An effective amount of disinfecting material is the amount sufficient to allow the disinfecting material to perform its action in reducing the number of micro-organisms existing on a given surface. Depending on the disinfecting material used, the amount used may be different.

The disinfectant cleaning compositions of the present invention may be applied directly on to the area to be treated or applied using a cloth or piece of material such as a spring device or aerosol can, a sponge, a brush or other mechanical/electrical device. The spraying device may be trigger operated or pump operated or electrically operated or operated by any such pressurized gas such as a can or a pressurizer. The spraying devices ensure uniform coverage of the area to be treated and maximizes the advantage of using liquid aqueous compositions.

The method for cleaning a substrate, therefore, includes contacting the disinfectant cleaning composition to a surface, for example a hard surface or a soft surface such as textile, fabric and/or upholstry, and optionally, rinsing the surface. In another embodiment the method further comprises the step of incorporating the composition into a wipe and contacting the wipe on the surface to be cleaned. In another embodiment, the method can further include applying energy to the substrate, and contacting the fabric with an absorbing material.

Various embodiments of the invention have been described. However, it should be understood that many variations and modifications may be made while remaining within the spirit and scope of the invention.

1. A disinfectant cleaning composition comprising:
from about 0.5% to about 6% by weight hydrogen peroxide;
from about 0.5% to about 20% by weight aloe vera and/or at least one antibacterial, antifungal and/or antiviral active component thereof; and
from about 99% to about 74% by weight of at least one solvent.

2. The disinfectant cleaning composition of claim 1, wherein the solvent is water.

3. The disinfectant cleaning composition of claim 1, wherein the disinfectant cleaning composition further comprises at least one fragrance.

4. The disinfectant cleaning composition of claim 3, wherein the fragrance is water soluble.

5. The disinfectant cleaning composition of claim 1, wherein the fragrance is present in a range from about 0.05% to 5% by weight based on the total weight of the disinfectant cleaning composition.

6. The disinfectant cleaning composition of claim 1, wherein the fragrance is selected from the group of: aromatic and aliphatic esters, aliphatic and aromatic alcohols, aromatic and aliphatic ketones, aromatic and aliphatic lactones, aromatic and aliphatic aldehydes, aromatic aldehydes, aliphatic and aromatic ethers, condensation products of aldehydes and amines, and mixtures thereof.

7. The disinfectant cleaning composition of claim 1, wherein the disinfectant cleaning composition comprises:
from about 1% to about 5% by weight hydrogen peroxide;
from about 1% to about 5% by weight aloe vera and/or at least one antibacterial, antifungal and/or antiviral active component thereof; and
from about 98% to about 80% by weight solvent.

8. A disinfectant cleaning composition comprising:
a fragrance, wherein the disinfectant cleaning composition comprises:
from about 0.5% to about 6% by weight hydrogen peroxide;
from about 0.5% to about 20% by weight aloe vera and/or at least one antibacterial, antifungal and/or antiviral active component thereof;
from about 0.05% to about 5% by weight fragrance; and
from about 98.95% to about 69% by weight solvent.

9. The disinfectant cleaning composition of claim 7, wherein the solvent is water.

10. The disinfectant cleaning composition of claim 8, wherein the solvent is water and the fragrance is water-soluble.

11. A disinfectant cleaning composition comprising:
from about 2% to about 4% by weight hydrogen peroxide;
from about 2% to about 10% by weight aloe vera and/or at least one antibacterial, antifungal and/or antiviral active component thereof;
from about 0.02% to about 2% fragrance; and
from about 95.8% to about 84% solvent.

12. The disinfectant cleaning composition of claim 11, wherein the solvent is water.

13. The disinfectant cleaning composition of claim 12, wherein the solvent is water and the fragrance is water-soluble.

14. A disinfectant cleaning composition comprising:
from about 0.5% to about 6% by weight hydrogen peroxide;
from about 0.5% to about 20% by weight aloe vera and/or at least one antibacterial, antifungal and/or antiviral active component thereof; and
from about 99% to about 74% solvent;
wherein the hydrogen peroxide is derived from one or more hydrogen peroxide producing sources.

15. The disinfectant cleaning composition of claim 1, wherein the at least one antibacterial, antifungal and/or antiviral active component is selected from one or more anthraquinones that are present in aloe vera.

16. The disinfectant cleaning composition of claim 7, wherein the at least one antibacterial, antifungal and/or antiviral active component is selected from one or more anthraquinones that are present in aloe vera.

17. The disinfectant cleaning composition of claim 11, wherein the at least one antibacterial, antifungal and/or antiviral active component is selected from one or more anthraquinones that are present in aloe vera.

18. The disinfectant cleaning composition of claim 14, wherein the at least one antibacterial, antifungal and/or antiviral active component is selected from one or more anthraquinones that are present in aloe vera.
